(19) United States
(12) Patent Application Publication
(10) Pub. No.: US 2019/0167738 A1
(43) Pub. Date: Jun. 6, 2019

(54) COMPOSITIONS CONTAINING EUGLENA GRACILIS FOR VIRAL PROTECTION AND RELATED METHODS

(71) Applicant: KEMIN INDUSTRIES, INC., Des Moines, IA (US)

(72) Inventors: Kelli HERRLINGER, Ankeny, IA (US); Joanne LASRADO, Des Moines, IA (US); Laura WONDERLING, Des Moines, IA (US); Yong ZHU, Norwalk, IA (US)

(21) Appl. No.: 16/165,712

(22) Filed: Oct. 19, 2018

Related U.S. Application Data
(60) Provisional application No. 62/593,590, filed on Dec. 1, 2017.

(51) Int. Cl. A61K 36/02; A61K 31/716; A61P 31/16; A61P 11/00

(52) U.S. Cl. CPC A61K 36/02; A61P 11/00; A61P 31/16; A61K 31/716

(57) ABSTRACT

The present invention relates to methods and compositions for preventing or treating a viral infection in animals or humans, including but not limited to administering whole cell Euglena gracilis and/or 95% Paramylon to an animal or a human as an immune stimulator. Another aspect of the present invention relates to reducing the severity and/or duration of symptoms associated with a viral infection.

### CROSS-REFERENCE TO RELATED APPLICATION

This application claims the benefit of priority to U.S. Provisional Patent Application No. 62/593,590, filed Dec. 1, 2017, entitled “EUGLENA GRACILIS AS AN ANTIVIRAL,” the entirety of which is incorporated herein by reference.

### BACKGROUND OF THE INVENTION

Influenza, commonly called “the flu,” is caused by viruses that infect the respiratory tract. Compared with most other respiratory infections, such as the common cold, the flu often causes a more severe illness.

Typical flu symptoms include fever (usually 100-103° in adults and often even higher in children) and respiratory symptoms, such as cough, sore throat, runny or stuffy nose, as well as headache, muscle aches, and often extreme fatigue. Although less frequent, nausea, vomiting, and diarrhea can sometimes accompany the flu, especially in children.

Most people who get the flu recover completely in one to two weeks, but some people develop serious and potentially life-threatening medical complications. Because each flu season is different in length and severity, the number of serious illnesses and deaths that occur each year varies. In the past 30 years, the annual death rate from flu-related causes has ranged from 3,000 to 49,000 deaths per year. Flu-related complications can occur at any age; however, very young children, pregnant women, the elderly, and people with chronic health problems are much more likely to develop serious complications of the flu than are younger, healthier people. Some of these severe complications can include bacterial pneumonia, ear infections, sinus infections, and worsening of chronic medical conditions, such as congestive heart failure, asthma, or diabetes.

Currently, the most frequent remedies for the flu are bed rest and drinking plenty of fluids. In some cases, the doctor will prescribe an antiviral medication, such as oseltamivir (Tamiflu), zanamivir (Relenza), or peramivir (Rapivab). While these antivirals can potentially shorten the illness by a day or two and may further prevent serious complications, their efficacy usually relies upon the patient starting treatment within 2 days of getting sick. These antivirals are often associated with side effects, including nausea and vomiting, dizziness, runny or stuffy nose, cough, diarrhea, and headache. Furthermore, oseltamivir has been associated with delirium and self-harm behaviors in teenagers.

Based upon the uncertainty about their effects combined with only a slight reduction in the time of illness, it is apparent that currently available antiviral drugs are not ideal options for treating influenza. An additional concern is that some strains of influenza are becoming resistant to antiviral drugs, and especially the older antivirals anoseltamivir, amantadine and rimantadine (Flumadine).

It is therefore a primary objective of the present invention to provide a better means and method of treating viral infections and, specifically, influenza infections.

### SUMMARY OF THE PRESENT INVENTION

The present invention relates to methods for improving and stimulating the immune system in response to a viral challenge, including but not limited to using a whole cell Euglena gracilis (WCE) for viral protection in humans or animals. WCE contains the soluble fraction and the paramylon (the beta[β]-glucan from Euglena). Another aspect of the present invention relates to the administration of a more purified paramylon (95%) from the whole cell Euglena gracilis, following viral, influenza-induced changes in human immune cell populations, using various known methods, dosages, and at various stages of infection. Another aspect of the present invention relates to methods for reducing the severity or minimizing the duration of symptoms associated with viral infections.

### BRIEF DESCRIPTION OF THE DRAWINGS

FIG. 1 is a chart comparing the effectiveness of algae-derived ingredients (95% or WCE) over YBG (yeast β-glucan) at increasing levels of CD3CD8 (Cytotoxic T Cells) following exposure of human peripheral blood mononuclear cells to viral stimulation.

FIG. 2 is a chart comparing the effectiveness of algae-derived ingredients (95% or WCE) over YBG at increasing levels of CD19CD69 (Activated B Cells) following exposure of human peripheral blood mononuclear cells to viral stimulation.

FIG. 3 is a chart comparing the effectiveness of algae-derived ingredients (95% or WCE) over YBG at increasing levels of CD19 Dectin 1 (B Cells with Dectin 1) following exposure of human peripheral blood mononuclear cells to viral stimulation.

FIG. 4A is a chart comparing the effectiveness of algae-derived ingredients (95% or WCE) over YBG at increasing levels of CD14CD69 (Activated Monocytes) following exposure of human peripheral blood mononuclear cells to viral stimulation.

FIG. 4B is a chart comparing the effectiveness of algae-derived ingredients (95% or WCE) over YBG at increasing levels of CD14CD69 (Activated Monocytes) following exposure of human peripheral blood mononuclear cells to viral stimulation.

FIG. 5 is a chart demonstrating superiority of effectiveness of the 95% over the YBG at increasing CD4CD25 (Regulatory T-cells) following exposure of peripheral blood mononuclear cells with virus.

FIG. 6 is a chart demonstrating superiority of effectiveness of the 95% over the YBG at increasing CD3CD8 (Cytotoxic T cells) following exposure of peripheral blood mononuclear cells with virus.

FIG. 7A is a chart demonstrating superiority of effectiveness of the 95% over the YBG at activating NK cells resulting in greater percentages of CD56CD69 (Activated NK cells) following exposure of peripheral blood mononuclear cells with virus.

FIG. 7B is a chart demonstrating superiority of effectiveness of the 95% over the YBG at activating NK cells resulting in greater percentages of CD56CD69 (Activated NK cells) following exposure of peripheral blood mononuclear cells with virus.

FIG. 8A is a chart demonstrating superiority of effectiveness of the 95% over the YBG at activating monocytes resulting in greater percentages of CD14CD69 (Activated Monocytes) following exposure of peripheral blood mononuclear cells with virus.

FIG. 8B is a chart demonstrating superiority of effectiveness of the 95% over the YBG at activating monocytes resulting in greater percentages of CD14CD69 (Activated Monocytes) following exposure of peripheral blood mononuclear cells with virus.

FIG. 9 is a chart demonstrating superiority of effectiveness of the 95% over the YBG at activating B cells resulting in greater percentages of CD19CD69 (Activated B cells) following exposure of peripheral blood mononuclear cells with virus.

FIG. 10 is a chart demonstrating superiority of effectiveness of the 95% over the YBG at increasing percentage of CD19 Dectin 1 (B cells with Dectin 1) following exposure of peripheral blood mononuclear cells with virus.

FIG. 11 is a study flow diagram (Example 2).

FIG. 12 depicts the total number of URTI episodes during run-in and after 30 and 90 days supplementation with whole cell Euglena or placebo (n=27).

FIG. 13 depicts the mean number of URTI episodes per person during run-in and after 30 and 90 days supplementation with whole cell Euglena or placebo (n=27). Values are mean±SD.

FIG. 14 depicts the number of URTI symptoms per person after 30 and 90 days supplementation with whole cell Euglena or placebo (n=27). Values are mean±SD.

FIG. 15 depicts the mean number of days with at least 1 reported URTI symptom per person as assessed by the WURSS-24 daily questionnaire during run-in and after 30 and 90 days supplementation with whole cell Euglena or placebo (n=27). Values are mean±SD.

FIG. 16 depicts the mean area under the curve (AUC) for WURSS-24 daily symptoms during run-in and after 30 and 90 days supplementation with whole cell Euglena or placebo (n=27). Values are mean±SD.

FIG. 17 depicts the mean number of sick days per person during run-in and after 30 and 90 days of supplementation with whole cell Euglena or placebo (n=27). Values are mean±SD.

### DETAILED DESCRIPTION OF EMBODIMENTS

The following description is merely exemplary in nature of the subject matter, and is not intended to limit the scope, application, or uses of any specific invention claimed in this application or in such other applications as may be filed claiming priority to this application, or patents issuing therefrom. Except in the examples or where otherwise expressly indicated, all numerical quantities in this description indicating amounts of material or conditions of reaction and/or use are to be understood as modified by the word “about” in describing the broadest scope of the invention.

The present invention relates to methods for improving and stimulating the immune system in response to a viral challenge, including but not limited to using a whole cell Euglena gracilis (WCE). The WCE contains the soluble fraction and the paramylon (the beta[β]-glucan from Euglena), as an antiviral, including but not limited to using the paramylon (β-glucan) fraction of the whole cell Euglena gracilis.

β-glucans are bioactive dietary fibers that consist of D-glucose monomers linked by a β-glycosidic bond. In the case of Euglena gracilis (an algae) derived β-glucans, this bond is primarily (>90%) a 1,3 linkage. β-(1,3) glucans are structural elements in the cell walls of bacteria, fungi including yeast, and algae, which the human immune system has evolved to identify as “non-self structures.” These “non-self structures” are recognized by pattern recognition receptors (PRRs) on host immune cells and also referred to as pattern associated molecular patterns (PAMP) (Inoue and Shinohara, 2014; Brown and Gordon, 2005). PRRs are present on several host immune cells including monocytes, macrophages, dendritic cells, neutrophils and natural killer (NK) cells. The primary PRRs for β-(1,3) glucans are Dectin-1, toll like receptors (TLR), complement receptor 3 (CR3), Lactosylceramide (LacCer) and Scavenger receptor (Brown, et al. 2003; Bacic, Fincher, and Stone, eds., 2009; Chan, et al. 2009). Recognition of the PRR by immune cells can induce both the innate and adaptive immune response and increase expression of the PRRs on the cell surface (Suresh, et al. 2013). Innate responses that can occur due to activation of these cellular populations include phagocytosis, oxidative burst, and cytokine/chemokine release while the adaptive immune response includes T-cell differentiation and priming of these T-helper cells as well as priming of cytotoxic T-cells and B cells. This activation can occur within the Peyer's patches as well as when these cells leave the Peyer's patches and travel to lymph nodes, spleen and bone marrow-leading to a systemic activation and priming of the immune response.

Since β-(1,3) glucans resist digestion, their oral ingestion results in their direct sampling from the gut lumen by macrophages and dendritic cells located within M cells or their uptake through M cells via endocytosis, phagocytosis, or transcytosis (Batbayar, et al. 2012). Hence, recognition of β-1,3-glucans triggers an immune response which is designed to protect the human body from the invading pathogen as part of the human immune system.

A few studies have reported the immunomodulation effects of Euglena gracilis, or its β-glucan, paramylon. Kondo et al. (1992) found a significant increase of peritoneal exudate cell IgM response in mice treated with 10 and 50 mg/kg paramylon, as well as significantly higher levels of lipopolysaccharide (LPS) induced IL-1 and IL-6 production. Russo et al. (2016) reported that Euglena paramylon can upregulate proinflammatory factors in lymphomonocytes. Furthermore, Bianchi et al. (2015) found that freshwater mussel fed with diets containing Euglena gracilis had increased phagocytic activity and tissue hemocyte accumulation with increased hemocyte viability upon E. coli challenge. The recent study conducted by Nakashima et al. (2017) also supported the effect of Euglena gracilis and paramylon on the immune system. Mice fed diets containing 2% Euglena gracilis or paramylon for 2 weeks before an influenza challenge had significantly increased survival rates compared to animals on a control diet. In addition, significantly higher levels of several cytokines as well as lower virus titers were found in the animals on the test diets compared to the controls.

Lipopolysaccharides (LPS) is a component of Gram-negative bacterial membranes (Zielen, et al. 2015). Influenza is a type of RNA virus that can cause respiratory infections (Doherty, et al. 2006). Since both LPS and influenza can induce immune responses, they have been used to mimic bacterial and viral infections, to investigate the immunomodulatory effects of extracts (Sohn, et al. 2015). Similarly, the immunomodulatory effects of yeast β-glucan using LPS in in vitro studies and the reduced incidence of cold symptoms in clinical trials are summarized by Stier et al. (2014).

Therefore, the objective of the study in Example 1 was to examine and compare the ex vivo effect of whole cell Euglena (WCE) and 95% Paramylon (95%, derived from Euglena) to yeast β-glucan (YBG) following viral, influenza-induced, changes in human immune cell populations.

The objective of Example 2 was to investigate the ability of whole cell Euglena gracilis (WCE) supplementation to augment immune function in normal health individuals, to decrease URTI incidence rates, and counter immune changes in healthy individuals, such as for instance endurance athletes.

According to at least one embodiment of the present invention, whole cell Euglena gracilis (WCE), 95% Paramylon, or combinations thereof, is administered to provide an immune response, for instance by activating immune cell populations.

According to at least one embodiment of the present invention, whole cell Euglena gracilis (WCE), 95% Paramylon, or combinations thereof, is administered to reduce the severity and/or duration of symptoms associated with the viral infection, including but not limited to reducing the days of upper respiratory tract symptoms, decreasing the total number of sick days or missed work days or missed training days, reducing the amount or frequency of medication use, URTI episodes, reducing symptom severity and/or duration.

According to at least one embodiment of the present invention, compositions containing whole cell Euglena gracilis (WCE), 95% Paramylon, or combinations thereof, are orally administered (including but not limited to soft-gel, capsule, tablet, gummies, powders, bars or other means of administration for supplement or functional foods and beverages). Oral administration of compositions containing whole cell Euglena gracilis (WCE), 95% Paramylon, or combinations thereof, would include but is not limited to doses that deliver from about 50 mg to 500 mg/day of (3-glucan, for instance about 100 mg to 300 mg/day of (3-glucan.

According to at least one embodiment, oral administration comprises administering compositions containing about 140 mg to 240 mg/day of (3-glucan; therefore, whole cell Euglena gracilis (WCE) would be about 250 mg to 500 mg/day and 95% paramylon would be about 145 mg to 255 mg/day.

According to certain embodiments, the compositions of the present invention are administered chronically. In alternative embodiments, the compositions of the present invention are administered at the first warning sign or onset of symptoms.

Example embodiments are provided so that this disclosure will be thorough, and will fully convey the scope to those who are skilled in the art. Numerous specific details are set forth such as examples of specific materials, compositions, and methods, to provide a thorough understanding of embodiments of the present disclosure. It will be apparent to those skilled in the art that specific details need not be employed, that example embodiments may be embodied in many different forms, and that neither should be construed to limit the scope of the disclosure. In some embodiments, well-known processes and well-known technologies are not described in detail. Equivalent changes, modifications and variations of some embodiments, materials, compositions, and methods can be made within the scope of the present technology with substantially similar results.

### EXAMPLE 1

Materials and Methods

Algae-derived ingredients or yeast β-glucan ingredients as described below:
1) Euglena whole cell extract [WCE]: Lot number 102616-AM-1; manufacturing date October 2016 (β-glucan content from certificate of analysis (CoA) 57.0%; megazyme analysis 51.1%), contains the β-glucan and the water-soluble fraction.
2) 95% Paramylon [95%] from Euglena: Lot number 06116-BG-1; manufacturing date June 2016 (β-glucan content from certificate of analysis (CoA) 95.8%, gravimetric analysis 97.7%). 3) Yeast β-glucan (YBG).

All extracts were prepared in order to deliver the same amount of β-glucan per well (dosed based on β-glucan content) as follows with 0.5 g per tube.
1) WCE with 50% β-glucan—2.5 ml of sterile phosphate buffered saline (PBS) was added to the tube containing 0.5 g
2) 95%-with 95% β-glucan—4.75 ml of sterile PBS was added to the tube containing 0.5 g
3) YBG extract with 67% β-glucan—3.35 ml of sterile PBS was added to the tube containing 0.5 g

After addition of the appropriate volume of PBS supplemented with 100 U/ml Penicillin, 100 mg/ml Streptomycin and 0.25 mg/ml Anti-Mycotic to achieve the stock concentration of active ingredient at 0.5 grams in 5 ml, each solution was vortexed for 30 seconds and allowed to sit at room temperature for one hour. One ml of each of solution was then diluted to 10 ml in sterile plain Roswell Park Memorial Institute (RPMI) medium supplemented with 100 U/ml Penicillin, 100 mg/ml Streptomycin and 0.25 mg/ml Anti-Mycotic and incubated in a 37° C./5% CO₂ incubator for one hour. After thoroughly vortexing, the solutions were added to the PBMCs at concentrations of 0.01, 0.1, 1, and 10 ul/mL (10 fold dilutions in all graphs left to right).

Buffy Coats (n=3) from normal healthy adult donors were obtained from the Stanford Blood Center and peripheral blood mononuclear cells (PBMCs) were isolated using Aragen's standard Histopaque separation protocols. PBMCs (1 million/ml) were suspended in RPMI culture medium supplemented with 10% fetal bovine serum (FBS), Penicillin/Streptomycin/Glutamax and stimulated with INFLUENZA (H1/N1 A/Puerto Rico/8/34; ATCC-VR-1469, Lot# 61465052; stock titer was 3.9e7 PFU/ml which was diluted to ˜3.3 ml in culture medium and added at 100 ml/well) in the presence/absence of the β-glucan ingredients added at 10-fold concentrations ranging from 10 ml/ml to 0.01 ml/ml (prepared as described above). Controls included untreated PBMCs (No Virus) with all concentrations of extracts, and PBMCs treated with virus alone (without extracts) and finally with lectin (PHA (5 mg/ml)) as a positive control. All will be set up in triplicate.

Cells were incubated for 24 hrs in a 37° C./5% CO₂ humidified incubator. After 24 hrs, cells were harvested in FACS tubes and stained with antibodies (as described below) and acquired on Attune N×T flow cytometer. Supernatants were harvested at 24 hrs and stored in the freezer to detect cytokines (e.g. IFN-γ). The analysis was performed using FlowJo analysis software and data were reported as percentages of cells in the appropriately gated populations. For the instrument/compensation settings, UltraComp Beads (ThermoFisher Scientic) will be used.

Zombie Aqua (Biolegend, Cat# 423101)—To identify viable cells

Anti-CD3 antibody (Biolegend, Cat# 300324)—To identify T cells

Anti-CD4 antibody (Biolegend, Cat# 00000)—To identify T helper subset of T cells

Anti-CD8 antibody (Biolegend, Cat# 301066)—To identify T cytolytic subset of T cells

Anti-CD19 antibody (Biolegend, Cat# 302206)—To identify B cells

Anti-CD14 antibody (Biolegend, Cat# 301834)—To identify Monocytes

Anti-CD56 antibody (Biolegend, Cat# 318328)—To identify NK cells

Anti-CD336 (NKp44) antibody (Biolegend, Cat# 325116)—To identify activated NK cells

Anti-CD69 antibody (Biolegend, Cat# 310942)—To identify activated cells-all cell types

Anti-CD25 antibody (Biolegend, Cat# 356138)—To identify activated cells-all cell types

Anti-CD282 (TLR2) antibody (Biolegend, Cat# 12810)—To identify cell surface receptor on monocytes

Anti-CD369 (Dectin 1) antibody (Biolegend, Cat# 355403)—To identify beta glucan receptor on B cells and Monocytes

Statistical Analysis

Upon completion of the study, analyzed data were compiled in a spreadsheet for analysis in GraphPad Prism. Between group comparison analysis were performed by a two-way repeated measures ANOVA of the stimulated cells (Virus+β-glucan ingredient) with the unstimulated (PBS+β-glucan ingredient) data subtracted for each donor then averaged for the three donors. Dunnett's multiple comparison was used for between group comparison at each concentration of β-glucan ingredient. In addition, the net Area Under the Curve (AUC) was calculated and compared by a one-way ANOVA between groups.

Results

For each cell population, comparisons were made between the algae-derived ingredients (95% or WCE) and YBG following exposure of human immune cells to viral stimulation.

Data showing WCE superiority to YBG

There were several cell populations that upon stimulation with Virus the response from the WCE appeared to be superior over the YBG. These cell populations are listed below.

Cells where WCE shows superiority to YBG

CD3CD8 (Cytotoxic T cells) (Table 1, FIG. 1)

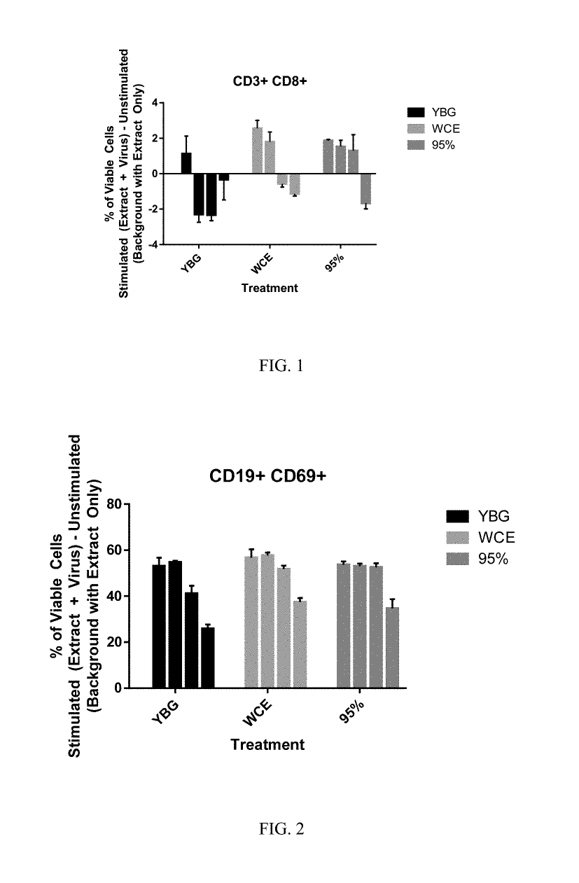

CD19CD69 (Activated B cells) (Table 2, FIG. 2)

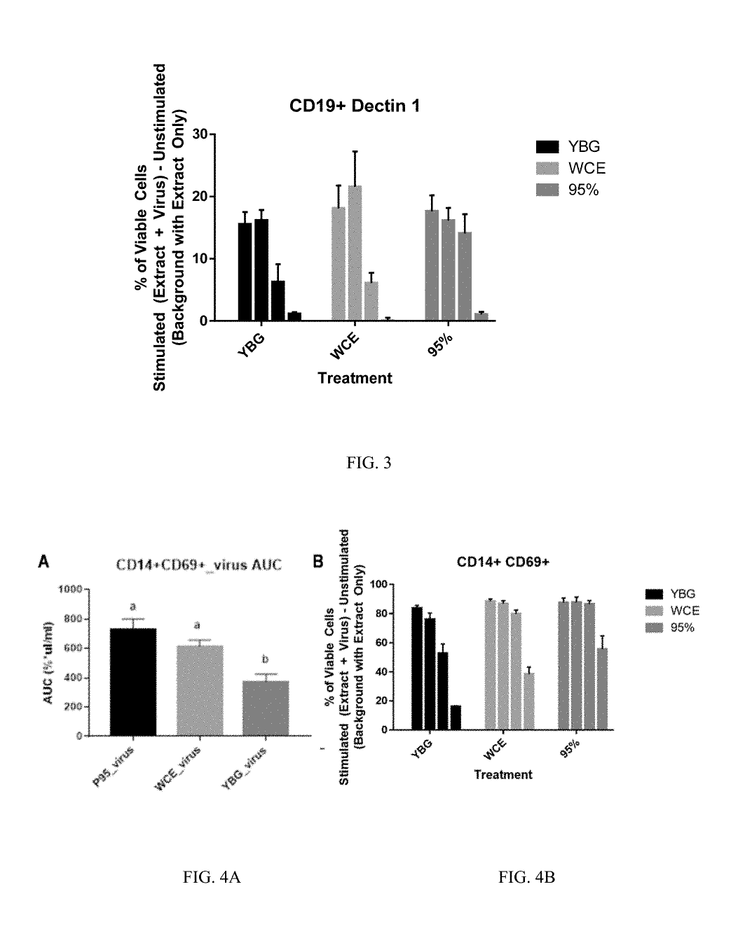

CD19 Dectin 1 (B cells with Dectin 1) (Table 3, FIG. 3)

CD14CD69 (Activated Monocytes) (Table 4, FIG. 4, A is the AUC data and B is the ANOVA data)

TABLE 1
Analysis of Cytotoxic T-cells (CD3 CD8) following
stimulation with Virus.

| | | Pairwise comparisons to | |
|---|---|---|---|
| | | Yeast Beta Glucan | Overall |
| ANOVA-all 4 | WCE | 0.1 p = 0.0014 | Treatment p = 0.0173 |
| concentrations | 95% | 0.1 p = 0.0023 | txt conc p = 0.0114 |
| | | 1 p = 0.0034 | |

TABLE 2
Analysis of Activated B-Cells (CD19 CD69) following
stimulation with Virus.

| | | Pairwise comparisons to | |
|---|---|---|---|
| | | Yeast Beta Glucan | Overall |
| ANOVA-all 4 | WCE | 1 p = 0.0005 and 10 | Txt conc. P = 0.0085 |
| concentrations | 95% | 1 p = 0.0003 and 10 | |

TABLE 3
Analysis of B-Cells with Dectin 1 (CD19 Dectin 1) following
stimulation with Virus.

| | | Pairwise comparisons to | | |
|---|---|---|---|---|
| | | Yeast Beta Glucan | Overall | Notes |
| ANOVA-all 4 | WCE | | NS but txt x | NS but use in patent to show |
| concentrations | 95% | | conc p = 0.1105 | WCE better than YBG trend |
| | | | | p = 0.1362 at 0.1 and 95% |
| | | | | better than YBG at 1 |
| | | | | (p = 0.0304) |

Activated monocytes were significantly different between WCE and YBG when evaluated both as analyzed as area under the curve and the repeated measures ANOVA.

TABLE 4
A is the Area Under the Curve and B is the data utilized in the ANOVA.
A

| CD14 + CD69 + cells | 95% | Whole cell | Yeast Beta |
|---|---|---|---|
| P = 0.0054 | Paramylon | euglena | glucan |
| 95% Paramylon | | P = 0.3917 | P = 0.0047 |
| Whole cell euglena | | | P = 0.0379 |
| Yeast Beta glucan | | | |

B

| | | Pairwise comparisons to | |
|---|---|---|---|
| | | Yeast Beta Glucan | Overall |
| ANOVA-all 4 | WCE | 0.1 p = 0.0458 | Treatment |
| concentrations | 95% | 1 p < 0.0001 | p = 0.0052 |
| | | at 10 | Txt x conc. |
| | | 0.1 p = 0.0322 | P = 0.0009 |
| | | 1 p < 0.0001 | |
| | | at 10 | |

There were several cell populations that upon stimulation with Virus the response from the 95% appeared to be superior over the YBG. These cell populations are listed below.

Cells where 95% shows superiority to YBG

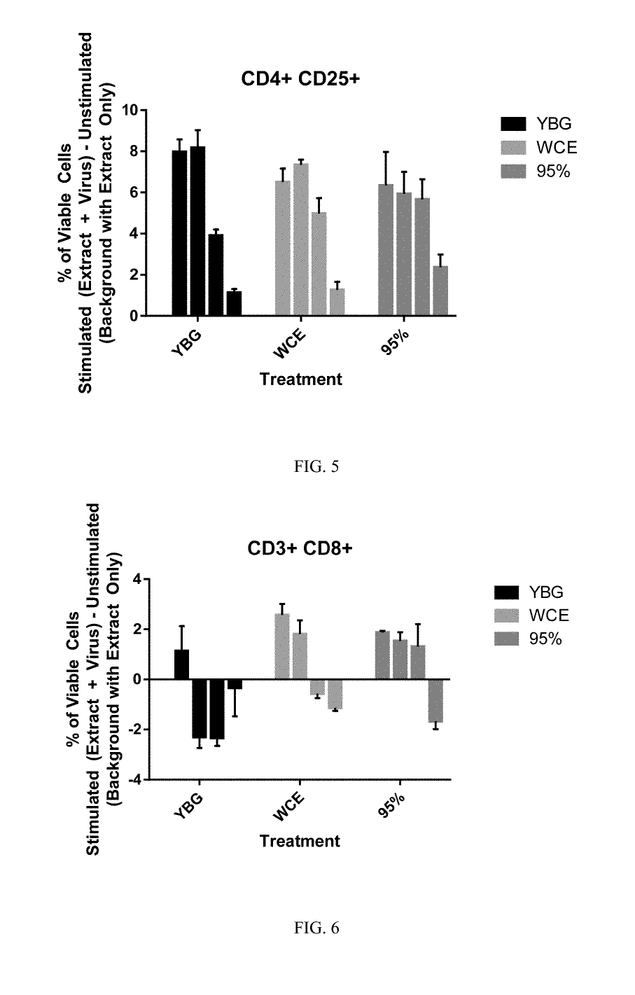

CD4CD25 (Activated T-helper cells also known as regulatory T-cells) (FIG. 5, Table 5)

CD3CD8 (Cytotoxic T cells) (FIG. 6, Table 6)

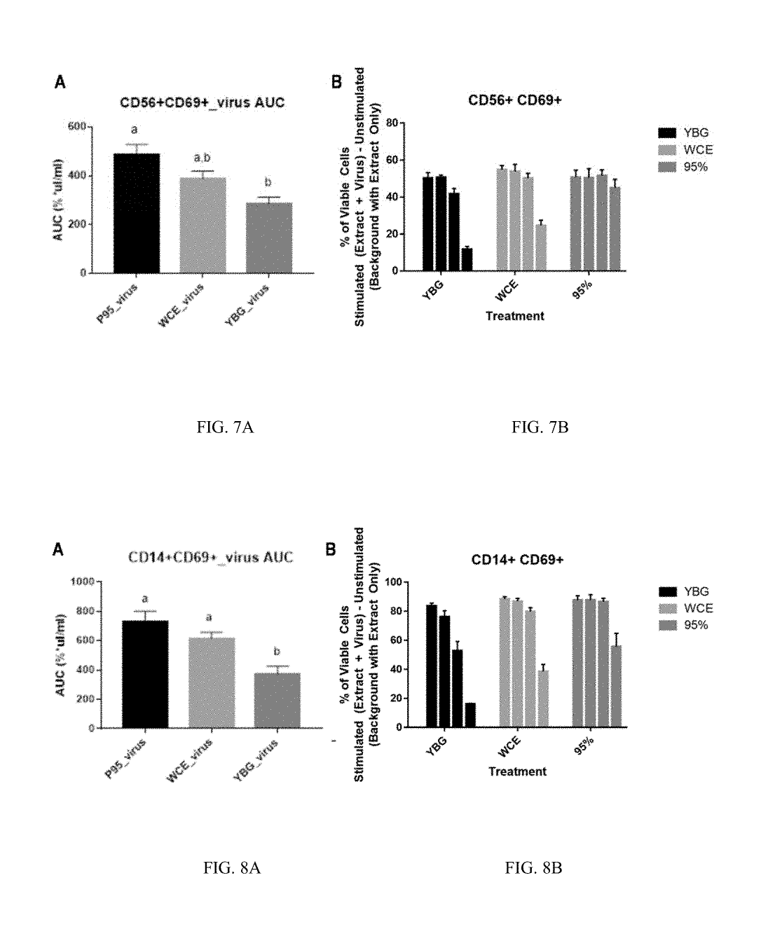

CD56CD69 (Activated NK cells) (FIG. 7, Table 7)

CD14CD69 (Activated Monocytes) (FIG. 8, Table 8)

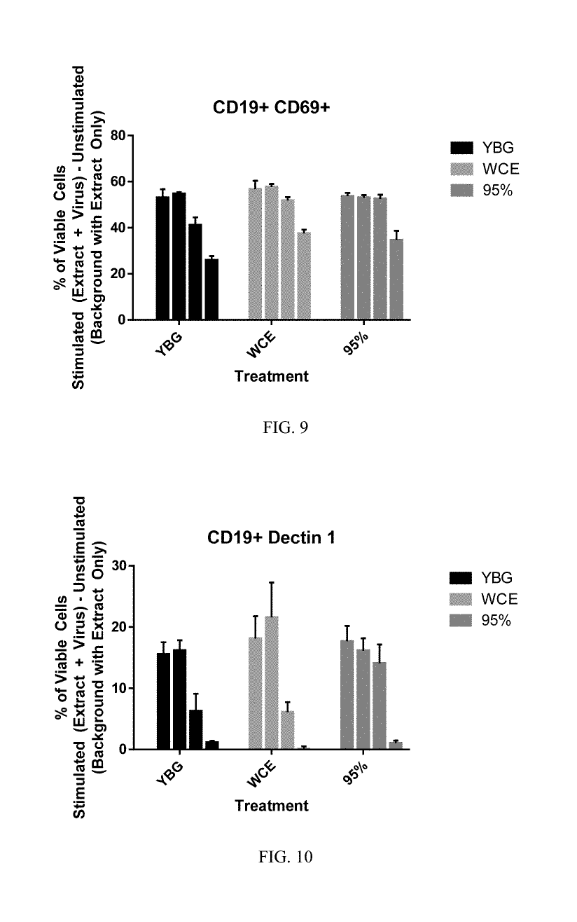

CD19CD69 (Activated B cells) (FIG. 9, Table 9)

CD19 Dectin 1 (B cells with Dectin 1) (FIG. 10)

TABLE 5

| | | Pairwise comparisons to | |
|---|---|---|---|
| | | Yeast Beta Glucan | Overall |
| ANOVA-all 4 | WCE | No different | Txt x conc. |
| concentrations | 95% | 0.01 p = 0.0494 | p = 0.0080 |
| | | 0.1 p = 0.0093 | |
| | | 1 p = 0.0342 | |

TABLE 6
Cytotoxic T-cell after stimulation with Virus.

| | | Pairwise comparisons to | |
|---|---|---|---|
| | | Yeast Beta Glucan | Overall |
| ANOVA-all 4 | WCE | 0.1 p = 0.0014 | Treatment p = 0.0173 |
| concentrations | 95% | 0.1 p = 0.0023 | txt x conc p = 0.0114 |
| | | 1 p = 0.0034 | |

TABLE 7A
Activated NK Cells after stimulation with Virus. A is the Area
Under the Curve and B is the data utilized in the ANOVA

| CD56 + CD69 + cells | 95% | Whole cell | Yeast Beta |
|---|---|---|---|
| P = 0.0091 | Paramylon | euglena | glucan |
| 95% Paramylon | | P = 0.1742 | P = 0.0071 |
| Whole cell euglena | | | P = 0.1454 |
| Yeast Beta glucan | | | |

TABLE 7B
Activated NK Cells after stimulation with Virus. A is the Area
Under the Curve and B is the data utilized in the ANOVA

| | | Pairwise comparisons to | |
|---|---|---|---|
| | | Yeast Beta Glucan | |
| ANOVA-all 4 | WCE | only at highest conc. | Treatment p = 0.0158 |
| concentrations | 95% | 1 p = 0.0277 | txt x conc p = 0.0003 |
| | | @10 | |

TABLE 8A
Activated Monoctyes after stimulation with Virus. A is the Area
Under the Curve and B is the data utilized in the ANOVA

| CD14 + CD69 + cells | 95% | Whole cell | Yeast Beta |
|---|---|---|---|
| P = 0.0054 | Paramylon | euglena | glucan |
| 95% Paramylon | | P = 0.3917 | P = 0.0047 |
| Whole cell euglena | | | P = 0.0379 |
| Yeast Beta glucan | | | |

TABLE 8B
Activated Monoctyes after stimulation with Virus. A is the Area
Under the Curve and B is the data utilized in the ANOVA

| | | Pairwise comparisons to | |
|---|---|---|---|
| | | Yeast Beta Glucan | |
| ANOVA-all 4 | WCE | 0.1 p = 0.0458 | Treatment p = 0.0052 |
| concentrations | 95% | 1 p < 0.0001 | Txt x conc. P = 0.0009 |
| | | at 10 | |
| | | 0.1 p = 0.0322 | |
| | | 1 p < 0.0001 | |
| | | at 10 | |

TABLE 9
Activated B Cells after stimulation with Virus ANOVA.

| | | Pairwise comparison to YBG | |
|---|---|---|---|
| ANOVA-all 4 | WCE | 1 p = 0.0005 and 10 | Txt x conc. |
| concentrations | 95% | 1 p = 0.0003 and 10 | P = 0.0085 |

Although not statistically significant a trend was observed for B cells (CD19 positive) with Dectin 1 (FIG. 10, p=0.1105). Pairwise comparison showed that at 0.1 ul/mL WCE trended to be superior to YBG at p=0.1362, and 95% was superior to YBG at 1 ul/mL (p=0.0304).

RESULTS:

In summary, stimulation with a Viral challenge resulted in effects on immune cell populations. Immune cell populations were identified where WCE or 95% were superior to YBG. Table 10 summarizes the observed findings when the PBMC treated with WCE, 95% or YBG were stimulated with the Virus.

TABLE 10
Summary of findings for Stimulation of PBMC with Virus.
Viral Stimulation

| Data indicative of WCE > YBG | Data Pattern Suggests | Data indicative of 95% > YBG |
|---|---|---|
| ↑ Cytotoxic T-cells | When stimulated with Virus, WCE/95% | ↑ Activated T-helper cells (with CD25) |
| ↑ Activated B-cells (CD69) | seem better than YBG for the following | ↑ Cytotoxic T-cells |
| ↑ B-cells with Dectin-1 expression | 1) Cytotoxic T-cells | ↑ Activated NK-cells (with CD69) |
| ↑ Activated Monocytes (CD69) | 2) Activation via increased CD69 expression | ↑ Activated B-cells (with CD69) |
| | a) B-cells | ↑ B-cells with Dectin-1 expression |
| | b) Monocytes | ↑ Activated Monocytes (CD69) |
| | c) NK-cells\hat{ } | |
| | 3) Increased Dectin 1 receptor | |
| | expression on B-cells | |

* Equivalence with YBG in WCE group only
\hat{ }Improvement in 95% over YBG only

### EXAMPLE 2

Description:

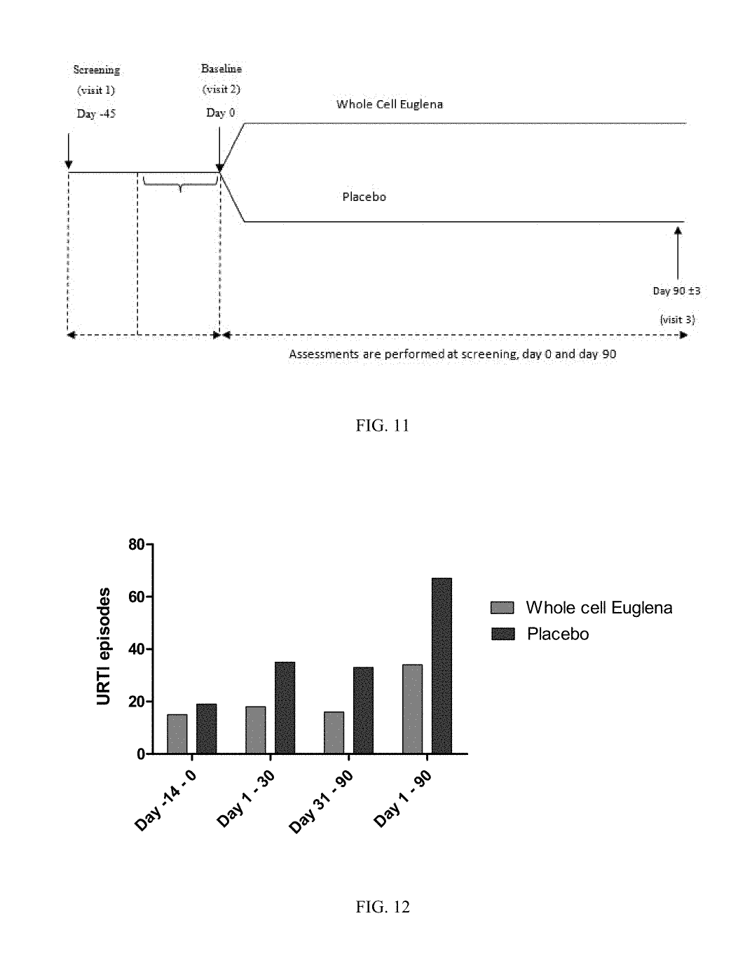

This was a randomized, double-blind, placebo-controlled, parallel study. The study consisted of a 90-day supplementation period, Study Flow Diagram in shown in FIG. 11. At screening, participants were deemed healthy by medical history and physical exam, vital signs, hematology and clinical chemistry. Participants were provided with and given instructions on completing the daily WURSS-24 questionnaire. Eligible participants returned for their baseline visit (Day 0) and were randomized into either the whole cell Euglena supplementation arm or the placebo arm. Participants returned to the clinic on Day 90 and vital signs and anthropometric measures were taken, blood was sampled for hematology and clinical chemistry and a product tolerability questionnaire was administered.

Enrollment Criteria:

Healthy male and female endurance athletes were enrolled into the study if they met all inclusion criteria and did not meet any exclusion criteria as described below.

Inclusion Criteria:
1) Males and females 21 to 65 years of age
2) Body Mass Index (BMI)>18 kg/m² to <35 kg/m².
3) Willingly complied with a wash-out period for nutritional supplements known to affect immune function and did not consume these supplements for the entire study period
4) Females of childbearing potential agreeing to use a medically approved method of birth control and had a negative urine pregnancy test result.
5) Agreed to maintain a consistent diet (including medications, vitamin and supplements not covered by the exclusion below) and lifestyle routine throughout the study
6) Agreed to abstain from exercising, tobacco use, and nutritional supplements on the morning of a study visit
7) Agreed to abstain from music, computer/cell phone use during clinic visits
8) Agreed to refrain from consuming candy, chewing gum, during in-clinic visits.
9) Agreed to abstain from consuming caffeinated beverages and other caffeine-containing products for 1 hour prior to and during clinic visits.
10) An endurance training athlete defined (as per Mach et al. 2017(1) and Gleeson et al. 2015(2)):
   a. Participated in an endurance (aerobic) sport for 1.5-3 hours/day for 5-6 days per week (e.g. cycling, running, triathlon, swimming, soccer, Nordic skiing, basketball, hockey, etc.)
11) Healthy as determined by laboratory results and medical history
12) Willingness to take supplement, complete questionnaires, records, and diaries associated with the study, some of which are daily, and to complete all clinic visits.
13) Has given voluntary, written, informed consent to participate in the study

Exclusion Criteria:
1) Women who were pregnant, breastfeeding, or planning to become pregnant during the course of the trial
2) Previous major gastrointestinal surgery (absorption of test product may be altered) or other digestive disorder which may have interfered with the absorption of nutrients including inflammatory bowel disease, irritable bowel syndrome, chronic constipation, and history of chronic diarrhea; history of surgery for weight loss, gastroparesis, or clinically important lactose intolerance; and chronic GI illness.
3) Consumed doses of beta-glucan-containing nutritional supplements (including algae, yeast or mushroom extracts), and was not willing to stop taking these supplements for 4 weeks prior to baseline and during the study
4) Chronic consumption of anti-inflammatory medications or medications known to affect immune function on a daily basis, including medications for allergies and asthma, within 4 weeks of visit 1 and within 48 hours of study visits during the study period (81 mg aspirin is acceptable)
5) Had an upper respiratory tract infection at baseline (visit will be rescheduled).
6) Taken antibiotics within 4 weeks of screening and during the study period
7) Diagnosed with a chronic inflammatory condition
8) Type I or Type II diabetes or clinically important renal, hepatic, cardiac, pulmonary, pancreatic, neurologic, or biliary disorder, or a recent history (prior 2 years) of cancer other than non-melanoma skin cancer.
9) Current use of antipsychotic medications such as clozapine, Risperdal, Abilify, Zyprexa or Seroquel within 4 weeks of visit 1
10) Chronic recurring respiratory signs and symptoms due to allergies (including seasonal allergies) or chronic bronchitis, asthma, or wheezing
11) Presence of auto-immune disorders
12) Chronic unusual sleep routine (examples: irregular routine with frequent late nights, studying, partying)
13) Use of immunomodulators (including corticosteroids) such as immunosuppressant or immunostimulant medications within 4 weeks of baseline and during the study period
14) Consumed >100% RDA (in supplement form) or used dietary supplements known to affect or taken with the intent of modulating immune function within 2 weeks of screening and during the study period
15) Chronic use of Antacids and Proton Pump Inhibitors (PPI).
16) Prebiotics and Probiotics unless on a stable regimen.
17) Unwilling to have blood drawn
18) History of diagnosed depression in the 2 years prior to screening
19) History of eating disorders or extreme dietary habits
20) Use of marijuana (any form of consumption) within the past 2 weeks and was unwilling to stop use for the duration of the study
21) Active infection or signs/symptoms of an acute infection at study visits. Test visits were re-scheduled to allow participant to be symptom-free of any type of acute systemic infection for at least 5 days prior to clinic visit
22) Heavy use of tobacco (defined as smoking more than 1 pack per week during past 3 months) or e-cigarettes
23) Consumption of ≥14 drinks per week or more than 4 standard alcoholic drinks/day for men and 3 standard alcoholic drinks/day for women (1 drink=12 oz. beer, 5 oz. wine, or 1½ oz. distilled spirits)
24) Alcohol or drug abuse within the last 2 years
25) Volunteers who planned to donate blood during the study or within 30 days of completing the study
26) Subjects who were not willing to comply with study procedures and study product or placebo consumption each day 30 minutes before breakfast on and empty stomach
27) Subject had a known allergy to the test material's active or inactive ingredients
28) Subjects with unstable medical conditions as assessed by the QI
29) Clinically significant abnormal laboratory results at screening as assessed by the QI
30) Participation in a clinical research trial within 30 days prior to randomization
31) Individuals who were cognitively impaired and/or who are unable to give informed consent
32) Any other condition which in the Investigator's opinion may have adversely affected the subject's ability to complete the study or its measures or which may pose significant risk to the subject

Study Products:

Treatment Arm:

Euglena Whole cell Extract

Active Ingredients: 367 mg Whole Cell Euglena, contains the (3-glucan and the water-soluble fraction.

Mode of Administration: Oral

Dose of administration: Intake of one capsule per day on an empty stomach

Placebo Arm:

Non-Active Ingredients: Microcrystalline cellulose, hypromellose, titanium dioxide, water

Mode of Administration: Oral

Dose of administration: Intake of one capsule per day on an empty stomach

Cold and Flu-like Symptom Assessment by WURSS-24 Questionnaire:

Evaluation of upper respiratory tract infection symptoms were assessed through daily

WURSS-24 questionnaire, the questionnaire asked about health status, presence and severity of URTI symptoms and symptoms related to allergy, as well as their impact on quality of life (18). A new URTI episode was defined as the sudden appearance of 1 or more symptoms not attributed to allergies with at least 2 days of ‘not sick’ in between as defined by Murdoch et al. (19). The WURSS-24 questionnaire was used to capture the incidence, frequency and severity of URTI symptoms and has been validated by Barret et al 2009.

Statistical Methods:

For continuous outcome variables, descriptive statistics were presented for the intervals of Run-in (Day -14 to Day 0), Days 1 through 30, Days 31 through 90, and Days 1 to 90. Differences between groups were assessed by the Chi-square or Fisher's exact (2-tail) test, as appropriate, ANOVA and ANCOVA.

All tests of significance were performed at alpha=0.05, 2-sided. Statistical analyses were conducted using SAS for Windows (version 9.3, Cary, N.C.).

Efficacy Data:

URTI episodes:

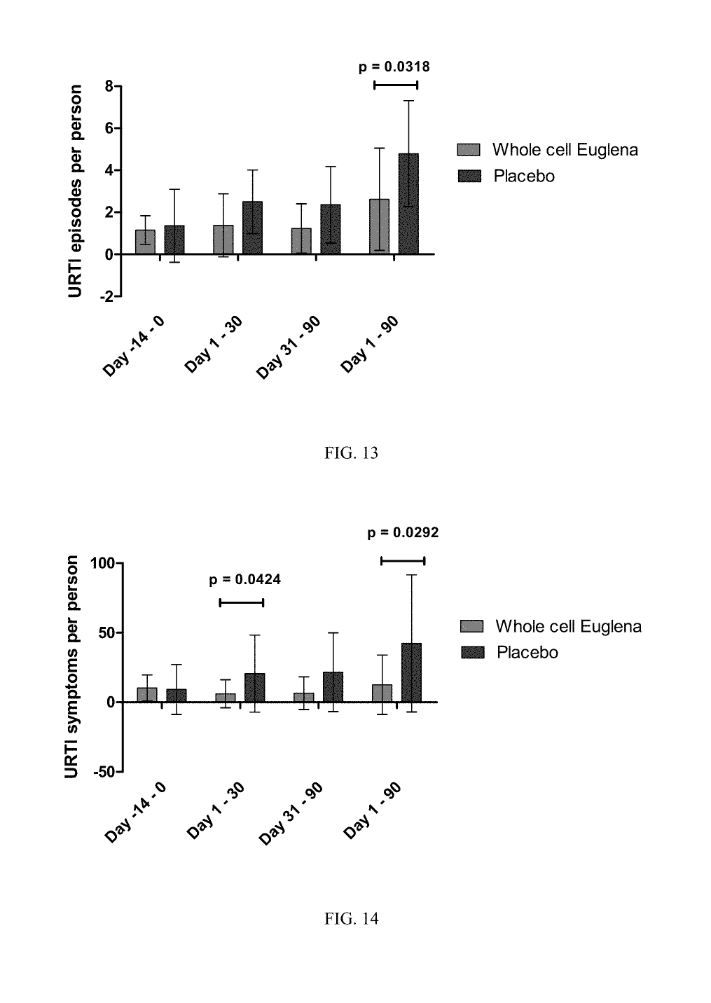

The number of URTI episodes was defined as the appearance of 1 or more symptoms not attributed to allergies with at least 2 days of “not sick” in between. Absolute number of URTI episodes reported by participants supplemented with whole cell Euglena was less (34 episodes) than those on placebo (67 episodes), FIG. 12. Participants supplemented with whole cell Euglena had 45% less URTI episodes per person compared to those taking placebo from Day 1 to Day 90 (FIG. 13, p=0.03).

TABLE 11
Total number of URTI episodes during run-in and after 30 and 90 days
supplementation with whole cell Euglena or placebo (n = 27)

| | Whole cell Euglena | Placebo |
|---|---|---|
| Study Day | N = 13 | N = 14 |
| Run-in (Day −14 to Day | 15 | 19 |
| 0) | | |
| Day 1 through Day 30 | 18 | 35 |
| Day 31 through Day 90 | 16 | 33 |
| Day 1 through Day 90 | 34 | 67 |

n, number;
SD, standard deviation;
Min, minimum;
Max, maximum;
* Between group p-value was generated from ANOVA with Group as a fixed effect.
(r) indicates values were ranked prior to generating ANOVA

URTI symptoms:

Participants taking whole cell Euglena reported a significantly lower number of URTI symptoms from Day 1 through 30 (p=0.04) and for Day 1 through 90 (p=0.03) compared to participants taking placebo. Between Day 1 and 90, participants taking whole cell Euglena reported 70% less symptoms than participants taking placebo (FIG. 14. 12.62 vs. 42.29 symptoms/person, p=0.03).

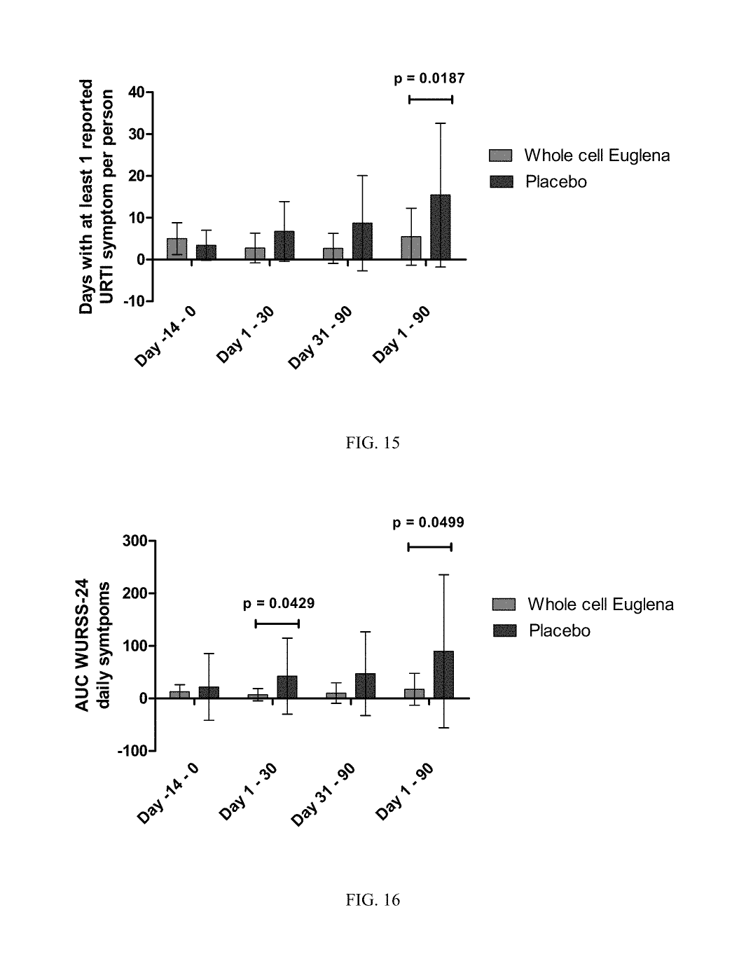

The mean number of days with at least 1 reported URTI symptom per person was also 65% lower in the group taking whole cell Euglena compared to those taking placebo between Day 1 and 90 (FIG. 15, p=0.02).

The global illness severity, as assessed by total AUC for the WURSS-24 daily symptoms, was reduced by 80% in participants supplemented with whole cell Euglena compared to placebo (FIG. 16, p<0.05). The mean WURSS-24 severity scores were not significantly different between groups after 30 and 90 days of supplementation.

Impact on daily life:

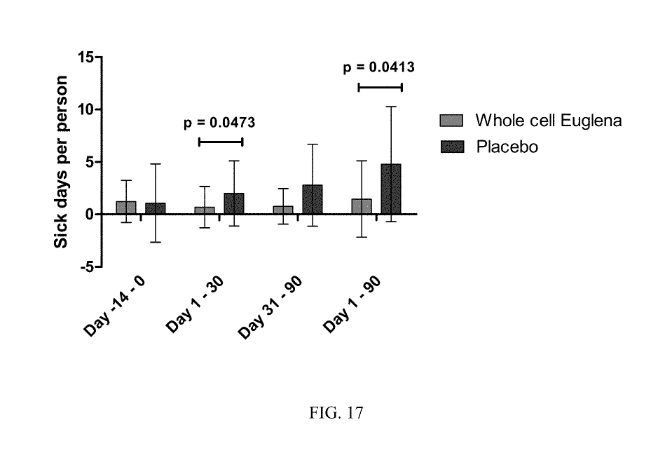

Sick days and cold medication use have an impact on daily life as well as athletic performance. Participants supplemented with whole cell Euglena had 70% fewer sick days than those taking placebo from Day 1 to 90 (FIG. 17, p=0.04). In general, individuals who consumed whole cell euglena have fewer incidences of cold medication use, missed work days, and missed training days.

TABLE 12
Incidence of Upper respiratory tract infection symptoms, cold
medication use, missed work days, and missed training days.

| | | Whole | |
|---|---|---|---|
| | | Cell | |
| | | Euglena | Placebo |
| Timeframe | Event | (n = 13) | (n = 14) |
| Day 0 | Total Number of Upper | 164 | 592 |
| through | Respiratory Tract | | |
| Day 90 | Infection Symptoms | | |
| | Episode duration days/per person | 1.89 | 3.20 |
| | Total Number of Common Cold | 6 | 22 |
| | Medication Use | | |
| | Total Number of Missed Work Days | 2 | 6 |
| | Total Number of Missed Training Days | 17 | 23 |

Conclusion:

The current study revealed the potential for 90 days of β-glucan supplementation to increase the non-specific, innate immune response in healthy individuals, including but not limited to endurance athletes. The ability of a natural health product such as whole cell Euglena to significantly reduce the total number of symptoms per person, number of episodes per person, global illness severity, the number of days with reported symptoms and number of sick days in athletes supports it use as a protective measure to lessen the burden of cold/flu symptoms in athletes, as well as healthy individuals generally.

The foregoing description and drawings comprise illustrative embodiments of the present inventions. The foregoing embodiments and the methods described herein may vary based on the ability, experience, and preference of those skilled in the art. Merely listing the steps of the method in a certain order does not constitute any limitation on the order of the steps of the method. The foregoing description and drawings merely explain and illustrate the invention, and the invention is not limited thereto, except insofar as the claims are so limited. Those skilled in the art that have the disclosure before them will be able to make modifications and variations therein without departing from the spirit and scope of the present invention. Accordingly, the drawings and detailed description are to be regarded as illustrative in nature and not restrictive in any way.

1. A method for stimulating an immune response in humans or animals comprising administering to humans or animals a composition comprising whole cell Euglena gracilis and/or 95% Paramylon.

2. (canceled)

3. The method of claim 1 whereby the Paramylon is derived from Euglena.

4. The method of claim 1 whereby the WCE is water soluble fraction only.

5. (canceled)

6. (canceled)

7. A composition for stimulating an immune response in animals or humans comprising an effective amount of water soluble fraction of whole cell Euglena gracilis and/or 95% Paramylon in a pharmaceutically acceptable carrier.

8. (canceled)

9. The composition of claim 7 whereby the Paramylon is derived from Euglena.

10. (canceled)

11. A method for manufacturing a composition for stimulating an immune response in animals or humans comprising adding whole cell Euglena gracilis 95% Paramylon, or combinations thereof, to a pharmaceutically acceptable carrier.

12. (canceled)

13. The method of claim 11 whereby the Paramylon is derived from Euglena.

14. A method of manufacturing an antiviral composition for humans or animals comprising adding water soluble fraction of whole cell Euglena gracilis and/or 95% Paramylon to a pharmaceutically acceptable carrier.

15. The method of claim 14 whereby the WCE is water soluble fraction only.

16. A method for reducing upper respiratory tract symptoms in humans or animals comprising administering to humans or animals an effective amount of whole cell Euglena gracilis and/or 95% Paramylon.

17. (canceled)

18. The method of claim 11 whereby the WCE is water soluble fraction only.

19. The method of claim 16 whereby the WCE is water soluble fraction only.

20. The method of claim 16 whereby the Paramylon is derived from Euglena.
